(19) United States
(12) Patent Application Publication
(10) Pub. No.: US 2010/0179091 A1
(43) Pub. Date: Jul. 15, 2010

(54) Treatment of Conditions Related to Shock

(75) Inventors: Geert W. Schmid-Schonbein, Del Mar, CA (US); Erik Kistler, South Boston, MA (US); John T. Watson, La Jolla, CA (US); Frank A. DeLano, San Diego, CA (US); Alexander H. Penn, San Diego, CA (US)

Correspondence Address: MOAZZAM & ASSOCIATES, LLC, 7601 LEWINSVILLE ROAD, SUITE 304, MCLEAN, VA, 22102, US

(73) Assignee: THE REGENTS OF THE UNIVERSITY OF CALIFORNIA, Oakland, CA (US)

(21) Appl. No.: 12/527,829

(22) PCT Filed: Feb. 20, 2008
(86) PCT No.: PCT/US08/54474

§ 371 Date: Apr. 1, 2010

Related U.S. Application Data
(60) Provisional application No. 60/902,085, filed on Feb. 20, 2007.
(60) Provisional application No. 60/913,790, filed on Apr. 24, 2007.

(51) Int. Cl. A61K 38/14; A61K 38/16; A61K 31/337; A61P 43/00

(52) U.S. Cl. 514/8; 514/12; 514/449

(57) ABSTRACT

Techniques are disclosed for prevention or treatment of physiological shock by administering a specific therapeutic agent or combination of therapeutic agents, which is/are able to use smaller volumes of reagent to achieve complete inhibition, than other previously described techniques.

This application is a U.S. national stage application under 35 U.S.C. 371 of the PCT application with Serial No. PCT/US2008/054474, filed Feb. 20, 2008, which claims priority to U.S. Provisional Patent Application Serial No. 60/902,085, filed Feb. 20, 2007, and to U.S. Provisional Patent Application Ser. No. 60/913,790, filed Apr. 24, 2007, the contents of all of which are hereby incorporated by reference in their entirety into this disclosure.

### GOVERNMENT INTERESTS

This invention was made with U.S. Government support under National Institute of Health (NIH) Grant No. HL67825. The government has certain rights in this invention.

### BACKGROUND OF THE INVENTION

1. Field of the Invention

The present invention relates to treatment of shock. In particular, the present invention relates to treatment of conditions related to shock.

2. BACKGROUND OF THE INVENTION

Shock is a life-threatening complication in situations associated with trauma including burns, surgery, ischemia, sepsis, radiation and other critical care applications. Shock is a broad term that describes a group of circulatory syndromes, all of which result in general microvascular and cellular dysfunction. Cell activation fundamentally alters the biomechanics of microvascular blood flow by a shift in rheological, adhesive, and cytotoxic cell properties. Cellular activation in the circulation can be detected by leukocytes or endothelial cells resulting in superoxide production, pseudopod projections, enzyme release, cytokine release, and expression of membrane adhesion molecules. The condition is accompanied by hypoxia, which leads to a depletion of the adenosine triphosphate (ATP), the failure of the sodium-potassium pump, mitochondrial dysfunction, and ultimately the release of a variety of toxic substances, including superoxides and other oxygen free radicals. Superoxides are toxic to essentially all tissues. They react with proteins and cause unfolding and are able to induce DNA damage. Eventually these stress responses give rise to irreversible cardiovascular collapse because of their combined effects on the microcirculation.

There are few satisfactory drugs, treatment methods, or interventions available for the prevention of conditions related to shock. Most currently available methods for the treatment of such conditions related to shock deal with the symptoms, rather than the cause. For this reason, current clinical approaches are limited in their efficacy and can only prevent further damage from occurring.

Thus, there is a need in the art for a more effective treatment of conditions related to shock. The treatment should be simple to administer, effective and capable of aiding in emergency situations.

### SUMMARY OF THE INVENTION

The present invention is a technique for treatment of conditions related to physiological shock by administering a more specific therapeutic agent or combination of therapeutic agents, which is/are able to use smaller volumes of reagent to achieve complete inhibition, than other previously described methods, for example, that in U.S. Pat. No. 6,534,283, which is incorporated by reference herein in its entirety. The present invention is based upon a new hypothesis for the cause of shock and multi-organ failure: self-digestion through gut ischemic complications rather than bacterial/endotoxin invasion.

In certain exemplary embodiments, the present invention is a technique for the prevention or treatment of physiological shock comprising administering to an individual containing an intestine a therapeutic dose of a serine protease inhibitor (and optionally a lipase inhibitor), directly into the small intestine to inactivate a protease (and a lipase), thereby ameliorating shock.

This technique aids in blocking formation of inflammatory mediators by pancreatic digestive enzymes in the intestine in septic shock and thereby reduces symptoms of multi-organ failure and significantly reduces mortality rate. It also serves to reduce morbidity and reduce post-operative complications, enhance recovery rate, and shorten hospital stays.

The treatment is administered into the lumen of the intestine to block fully activated digestive enzymes and auto-digestion of the intestine. The treatment is highly effective to attenuate prolonged formation of inflammation in septic shock, to attenuate destruction of the intestinal epithelial lining, and to reduce mortality. There is currently no comparable treatment for septic shock.

In one exemplary embodiment, the present invention is a method for pancreatic protease inhibition to reduce complications and hospital stay after radiation exposure, trauma, surgery or other injury. This includes a method for prevention or treatment of inflammatory symptoms and related complications (swelling, etc.) from surgical procedures comprising administering to an individual containing an intestine a therapeutic dose of a pancreatic enzyme inhibitor (and, optionally, lipase inhibitor) directly into the lumen of the small intestine. The emphasis here is to reduce post-operative patient complications and recovery periods by blockage of digestive enzymes during anesthesia and surgery. This will apply to any surgery (other than minor outpatient surgery), and maybe even in some cases of child birth. The blockade involves proteases, lipases, and possibly other pancreatic and bacterial enzymes in the intestine as well as enzymes that may have leaked into the peritoneum, the central circulation and into the lymphatics.

In another exemplary embodiment, the present invention is a method for pancreatic protease inhibition in septic shock. This includes a method for prevention or treatment of septic shock comprising administering to an individual containing an intestine a therapeutic dose of a pancreatic enzyme inhibitor (and, optionally, lipase inhibitor) directly into the small intestine to inactivate digestive enzymes, thereby ameliorating shock. The major enzymes at this stage are pancreatic enzymes, pancreatic lipases, and possibly bacterial enzymes. There is little evidence that treating amylases or nucleases can affect survival. However, they may be useful to protect against other cellular damage mechanisms. This invention is specific to septic shock, as opposed to the broader field of physiological shock.

In yet another exemplary embodiment, the present invention is a method for pancreatic lipase inhibition to reduce mortality after shock. This includes a method for prevention or treatment of physiological shock or inflammation comprising administering to an individual containing an intestine a therapeutic dose of a pancreatic lipase inhibitor directly into the small intestine to inactivate digestive enzymes, thereby ameliorating shock. The pancreatic lipase inhibitor is a different class of inhibitors from serine protease inhibitors (as described in the U.S. Pat. No. 6,534,283) and may be effective to prevent inflammatory mediator formation.

### DETAILED DESCRIPTION OF THE INVENTION

This invention describes techniques for treatment of conditions related to shock. Various exemplary embodiments are presented to provide a broad spectrum of treatment available and application to such related conditions.

As discussed above, the strategy for inhibiting gut enteral function has been described in U.S. Pat. No. 6,534,283, which is incorporated by reference herein in its entirety. This patent describes the use of protease inhibition in the lumen of the gut in principle and more specifically using specific commercially available protease inhibitors. The current strategy proposes numerous applications related to pancreatic protease inhibitors.

Death from heart, lung and kidney failure during shock due to inadequate blood flow can be prevented by an unusual experimental treatment that inhibits the aggressive enzymes that are produced in body to digest food.

The present invention provides evidence from animal studies that for the first time, studies showed that blockade of the digestive enzymes during shock leads to long-term survival. The results show a dramatic reduction of mortality in hemorrhagic, septic and secal ligation shock induced multi-organ failure. This treatment holds great promise for future clinical application, particularly in emergency rooms and before high-risk surgeries. When a person is in shock, his or her life is on the line. The patient's survival may be in jeopardy not just that day, but within an hour because healthy organs can fail and die in rapid succession.

An estimated 1 million cases of various types of shock are treated annually in U.S. hospital emergency rooms. Shock and sepsis are serious medical conditions with a fatality rate of approximately 29%. While the optimal management of shock patients can improve survival rates, overall shock remains a condition with a high death rate.

Administering a drug to inhibit the body's digestive enzymes is a relatively new approach that was begun in the past decade. In 1998 a finding was made in laboratory studies on the body's inflammatory cascade and the factors that turn this normal tissue-healing biological process into a virulent, out of control firestorm against the body's normal tissue.

The present invention is based on the latest research using rodent models of human hemorrhagic shock. Here it has been discovered that the sudden lowering of blood pressure that occurs in people suffering from stroke can provoke the body's digestive enzymes to break down the body's own intestinal and other tissues as if it were food. Such enzymes' abnormal actions may be defined as “auto-digestion.” Auto-digestion is dangerous because not only does it injure healthy tissue but also contributes to multi-organ failure, which can be fatal.

The healthy cells of the animals' intestinal tissue react to auto-digestion by releasing a slew of substances that can be toxic to the heart and other body organs. These substances, termed cytotoxic mediators, can reach these body organs via the blood stream. In recent studies, shock was induced in 19 lab rodents, all of which were then treated with therapies that mirror the emergency room care given to many human patients who suffer shock, which typically occurs when blood flow to the heart, lungs and other body organs is slowed as a result of trauma, dehydration, heart attack, stroke or radiation.

A total of 10 of the 19 lab rodents in shock were also treated with the experimental digestive enzyme inhibitor called ANGD. Eight of the ten survived. However, only one of the nine “untreated” animals in shock survived. The other eight animals died from organ failure within 12 hours. Although these “untreated” animals did not receive ANGD, the inhibitor, they were given basic shock care. The enzyme inhibitor ANGD dramatically improved the survival rate among the lab animals in which shock had been induced.

Dramatic improvements of survival were also shown in other models, including in an endotoxic shock (sepsis) model and in a cecal ligation shock model. Survival statistics in these tested models are shown in the tables below.

Table 1A shows that long-term survival following hemorrhagic (hypovolumetric) shock was significantly higher in the treated rather than the untreated animals after intestinal enzyme blockade. Similarly, long-term survival following endotoxic shock was significantly higher in the treated rather than untreated animals after intestinal enzyme blockade, as shown in Table 1B. Finally, Table 1C shows that in models exposed to septic shock through cecal ligation, long-term survival was significantly higher in the treated rather than untreated animals after intestinal enzyme blockade. These results show that the methodology according to the present invention serves to protect the animal and promote the survival thereof, even after various forms of shock are administered.

TABLE 1A
Long-Term Survival following Hypovolumetric Shock

| | # | Non-Survivor | Survivor | % Survivor |
|---|---|---|---|---|
| Control | 12 | 9 | 3 | 25% |
| Treated | 12 | 2 | 10 | 83% |

TABLE 1B
Long-Term Survival following Septic Shock - Endotoxin (LPS)

| | # | Non-Survivor | Survivor | % Survivor |
|---|---|---|---|---|
| Control | 13 | 9 | 4 | 31% |
| Treated | 11 | 1 | 10 | 91% |

TABLE 1C
Long-Term Survival following Septic Shock - Cecal Ligation

| | # | Non-Survivor | Survivor | % Survivor |
|---|---|---|---|---|
| Control | 10 | 9 | 1 | 10% |
| Treated | 10 | 1 | 9 | 90% |

In all of these studies treatment by pancreatic enzyme blockade was one hour after induction of shock. In hemorrhagic shock and in endotoxic shock (Tables 1A and 1B) blockade was only in the lumen of the intestine. In cecal ligation shock (Table 1C) blockade was also in the peritoneum surrounding the intestine.

In pig studies, experiments have been conducted to identify the time period when the experimental treatment will be the most effective in saving lives. The findings are relevant to the emergency care of human patients in shock. Data indicate that the earlier the treatment occurs, the better the chances for survival. Current research indicates that the window of opportunity for the treatment to be effective does not seem to be very narrow.

The discovery of the “auto-digestion” process and their positive findings from the experimental treatment ANGD are based on National Institutes of Health funded basic research to determine the origin of the inflammatory cascade that causes organ failure and death. Basically, inflammation is the body's mechanism to repair and to heal tissue. But in shock, the inflammation never stops. It is out of control. Normally the body senses when the inflammatory process has completed its job and brings it to a halt.

There is little surprise that tissue can be severely damaged by the actions of the body's digestive enzymes, which are secreted by the pancreas but do not become activated until they arrive into the intestines. Digestive enzymes have to be very aggressive, and there has to be a large quantity of them, for the body to efficiently digest, or break down, the food that we eat. Normally the intestinal tissue is protected from these enzymes by a layer of secreted mucus and by the tight packing of the cells in the intestinal wall. The enzymes are too big to defuse between these cells under normal conditions. However, problems arise under shock conditions when such enzymes are able to traverse these natural barriers and cause damage to the underlying tissue. The present invention is a way to alleviate or minimize the potential damage caused by this improper movement and destruction of the body's tissue by its own digestive enzymes.

In an exemplary embodiment, the present invention involves the administration of a pancreatic enzyme inhibitor directly into the lumen of the intestine by various techniques, including, for example by oral administration, introduction via an esophageal catheter, or direct injection into the lumen of the intestine during surgery. The agents that may be used individually or in combination include but are not limited to: Futhane (0.1-0.5 M), Foy (0.1-0.5M), Anti-alpha trypsin ( . . . ), Trasylol (Aprotinin, Bayer) (1.4 mg/ml), Cyclokaprone (100 mg/ml) as serine protease inhibitors; Orlestat (5 to 50 mg/ml), Lipostatin as lipase inhibitors; plus any other pancreatic enzyme inhibitor.

The amount administered is adjusted according to intestine size to achieve complete blockade of digestive enzyme activity. The blockade of the pancreatic enzymes includes use of protease inhibition or a combination of protease inhibition and lipase inhibition.

Useful examples of amount or duration for treatment are dependent on the particular type of animal (or human), its size, weight and other pertinent conditions of the animal (or human) being treated. In certain experiments, about 15 to 20 ml of concentrate enzyme inhibitor are given along the length of the rat intestine (which is about 90 cm long). In the pig, about 1 to 2 gallon of protease inhibitor may be given. The treatments may be given with protease inhibitor alone or in combination with a lipase inhibitor (as long as they are being given simultaneously). When given in combination, the drugs should be in the intestine at the same time for optimal treatment. The amounts and duration needed for beneficial results are essentially the same for all treatments described herein and throughout this disclosure. Thus, these variables carry through to the different conditions described below.

It is hereby demonstrated that blockade of pancreatic enzymes in the lumen of the intestine attenuates inflammatory symptoms after administration of a lethal dose of endotoxin (6 mg/kg). Experiments demonstrate reduced long-term mortality in the same sepsis model.

It is further demonstrated that the combination of 0.45 M Futhane and 5 mg/ml Orlestat gives significantly reduced mortality in a model of septic shock with near 100% mortality in the untreated control group.

The present invention may be used in numerous medical treatments, including but not limited to, treatment for prevention of multi-organ failure and mortality in septic shock. Any lipase inhibitor in combination with a pancreatic or leukocyte derived protease inhibitor may have utility to prevent inflammation in septic shock.

In another exemplary embodiment, which may be used for treatment for prevention of post-operational complications, including multi-organ failure, sepsis, morbidity and mortality, pancreatic protease inhibition is initiated to reduce complications and hospital stay after trauma/surgery/radiation exposure. Here, it has been shown that pancreatic enzymes in the intestine have the ability to generate powerful inflammatory mediators and that blockade of pancreatic enzymes in the lumen of the intestine attenuates inflammatory symptoms after different shock models.

In this embodiment, the present invention allows a reduction in inflammatory symptoms and complications (swelling, embolism formation, selected organ dysfunction, pulmonary embolism, incidence of stroke, patient mobility, morbidity, multi-organ failure, mortality) in any form of elective surgery/general anesthesia associated with elevated risks (such as prolonged surgery procedures, surgery with bypass requirements, surgery on patients with preconditions and risk factors, surgery involving the intestine and pancreas). This results in a reduction in post-surgical complications, enhance wound healing, reduce total recovery period, and reduce hospitalization requirements and time.

In elective surgery, pre-administration of a pancreatic enzyme inhibitor may be conducted directly into the lumen of the intestine by various techniques, including for example, by oral administration, introduction via an esophageal catheter, or direct injection into the lumen of the intestine during surgery. The agents that may be used are individually or in combination: Futhane (0.1 mM); Trasylol (Aprotinin, Bayer) (1.4 mg/ml), serine protease inhibitor; Orlestat (5 to 50 mg/ml), lipase inhibitor plus any other pancreatic enzyme inhibitor. The amount administered is adjusted according to intestine size and content to achieve complete blockade of digestive enzyme activity. As treatment, the inhibitor is administered after trauma or sepsis associated with risk for shock and multi-organ failure. As pretreatment the inhibitor is administered prior to general anesthesia/surgery.

This is the first intervention against a major source of inflammation in multi-organ failure associated with surgery/general anesthesia. Blockade of digestive enzymes prior to general anesthesia may serve to preserve barrier properties of the intestinal mucosa, reduce inflammation in the central circulation, and consequently reduce recovery and wound healing periods, post-surgical complications, hospital stays, etc.

A potentially important application of the digestive enzyme inhibition as pre-treatment is for patients subjected to radiation or chemotherapeutic treatment. It could also work for radiation treatment under other circumstances to reduce symptoms of multi-organ failure.

In yet another exemplary embodiment, the present invention provides a method for pancreatic protease inhibition in septic shock. There are many uses for this embodiment, including but not limited to, treatment for prevention of multi-organ failure and mortality in septic shock. Such treatment works by blocking formation of inflammatory mediators by pancreatic digestive enzymes in the intestine in septic shock and thereby reducing symptoms of multi-organ failure and mortality.

The treatment is administered into the lumen of the intestine to block fully activated digestive enzymes and auto-digestion of the intestine. The treatment is highly effective to attenuate prolonged formation of inflammation in septic shock, destruction of the intestinal epithelial lining, and reduces mortality.

It is demonstrated that blockade of pancreatic enzymes in the lumen of the intestine attenuates inflammatory symptoms after administration of a lethal dose of endotoxin (6 mg/kg). Experiments demonstrate reduced long-term mortality in the same sepsis model.

Administration of a pancreatic enzyme inhibitor may be conducted directly into the lumen of the intestine through various techniques, including, for example, by oral administration, introduction via an esophageal catheter, direct injection into the lumen of the intestine during surgery. The agents that may be used are individually or in combination: Futhane (0.1 mM); Trasylol (Aprotinin, Bayer) (1.4 mg/ml), serine protease inhibitor; Orlestat (5 to 50 mg/ml), lipase inhibitor; plus any other pancreatic enzyme inhibitor. The amount administered is adjusted according to intestine size to achieve complete blockade of digestive enzyme activity.

In another exemplary embodiment, the present invention is used for pancreatic lipase inhibition to reduce mortality after shock. This embodiment is very useful for developing treatment for prevention of multi-organ failure and mortality in hemorrhagic shock, preventive treatment to reduce the probability for development of multi-organ failure in elective surgery, long-term treatment to reduce production of lipid derived inflammatory mediators associated in chronic diseases. It is also particularly useful because there does not appear to be any treatment proposed to attenuate inflammation by blockade of lipase activity in the intestine in either acute or chronic inflammatory conditions.

This embodiment is designed as an intervention to block the lipase activity in the lumen of the intestine and also in the general circulation in those cases in which lipase enters from the lumen of the intestine into the circulation. This prevents formation of lipid derived inflammatory or cytotoxic mediators in shock and other inflammatory diseases and attenuate multi-organ failure in shock and chronic inflammation in diseases like hypertension, diabetes, the metabolic syndrome, cancers and in chronic degenerative diseases.

Recent evidence resulting in this invention suggests that a major component of inflammatory mediators from the intestine in shock causing multi-organ failure and mortality (e.g., after surgery/general anesthesia, trauma, radiation exposure, chronic diseases and any other condition leading to multi-organ failure) is derived from the action of pancreatic lipases (lipid splitting enzymes). Blockade of pancreatic lipase serves to reduce mortality during shock and reduce inflammation that leads to multi-organ failure. Blockade of pancreatic lipase prior to general anesthesia may serve to preserve barrier properties of the intestinal mucosa, reduce inflammation in the central circulation, and consequently reduce recovery and wound healing periods, post-surgical complications, hospital stays, etc.

The inventors have shown that the ischemic intestine produces a powerful set of lipid derived cytotoxic mediators and that the blockade of lipase in the intestine under in-vitro conditions blocks the production of lipid-derived cytotoxic mediators. Thus, the present invention serves to treat or decrease the damages caused by this finding.

The above exemplary embodiments have shown various uses and techniques for decreasing certain conditions related to shock. Thus, as a whole, the present invention is based on data from animal studies that show dramatic reduction in life-threatening shock by inhibiting a body's own aggressive digestive enzymes. This novel approach targets trigger mechanisms in auto-digestion before it launches lethal inflammatory cascade.

The following references, some as cited above, are hereby incorporated by reference in their entirety into this disclosure:
1. Schmid-Schonbein G W, Hugli T E. A New Hypothesis for Microvascular Inflammation in Shock and Multiorgan Failure: Self-Digestion by Pancreatic Enzymes. Microcirculation. 2005; 12:71-82.
2. Doucet J J, Hoyt D B, Coimbra R, et al. Inhibition of enteral enzymes by enteroclysis with nafamostat mesilate reduces neutrophil activation and transfusion requirements after hemorrhagic shock. J Trauma. 2004;56:501-511.
3. Fitzal F, DeLano F A, Young C, Schmid-Schonbein G W. Improvement in early symptoms of shock by delayed intestinal protease inhibition. Arch Surg. 2004;139:1008-1016.
4. Deitch E A, Shi H P, Lu Q, et al. Serine proteases are involved in the pathogenesis of trauma-hemorrhagic shock-induced gut and lung injury. Shock. 2003;19:452-456.
5. Shi H P, Liu Z J, Wen Y. Pancreatic enzymes in the gut contributing to lung injury after trauma/hemorrhagic shock. Chin J Traumatol. 2004; 7:36-41.
6. Muhs B E, Patel S, Yee H, et al. Inhibition of matrix metalloproteinases reduces local and distant organ injury following experimental acute pancreatitis. J Surg Res. 2003;109 :110-7.
7. Rosario H S, Waldo S W, Becker S A, et al. Pancreatic trypsin increases matrix metalloproteinase-9 accumulation and activation during acute intestinal ischemia-reperfusion in the rat. Am J Pathol. 2004;164:1707-16.
8. Fitzal F, DeLano F A, Young C, Rosario H S, Junger W G, Schmid-Schönbein G W. Pancreatic enzymes sustain systemic inflammation after an initial endotoxin challenge. Surgery, 134:446-456, 2003.
9. Penn, A H, Hugli, T E, Schmid-Schönbein, G W. Pancreatic enzymes generate cytotoxic mediators in the intestine. Shock, 27:296-304, 2007.
10. Penn, A H, Schmid-Schönbein, G W. Intestinal Necrosis in Shock: Free Fatty Acid Cytotoxicity and Degradation of Lipid-binding Proteins. In Press, 2008. Included as part of provisional application 60/913,790 filed on Apr. 24, 2007.
11. Penn, A H, Schmid-Schönbein, G W. The Intestine as Source of Cytotoxic Mediators in Shock: Free Fatty Acids and Degradation of Lipid-binding Proteins. AJP, in Press, 2008.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method for prevention or treatment of physiological shock, the method comprising:
administering to an individual containing an intestine a therapeutic dose of a pancreatic protease inhibitor into the small intestine to inactivate a protease, thereby ameliorating shock.

2. The method of claim 1, further comprising:
administering to the individual a therapeutic dose of a lipase inhibitor into the small intestine.

3. The method of claim 2, wherein the pancreatic protease inhibitor and the lipase inhibitor are administered simultaneously.

4. The method of claim 2, wherein the pancreatic protease inhibitor and the lipase inhibitor are administered sequentially.

5. The method of claim 1, wherein the protease is species-specific.

6. The method of claim 1, wherein the protease is a pancreatic protease.

7. The method of claim 1, wherein antioxidants are administered in conjunction with the plasma protease inhibitor.

8. The method of claim 1, wherein the plasma protease inhibitor is additionally administered orally.

9. The method of claim 1, wherein the plasma protease inhibitor is additionally administered intravenously.

10. A method for reducing complications after radiation, trauma or surgery, the method comprising:
administering to an individual containing an intestine a therapeutic dose of a pancreatic protease inhibitor into the small intestine to inactivate a protease, thereby ameliorating shock.

11. The method of claim 1, further comprising:
administering to the individual a therapeutic dose of a lipase inhibitor into the small intestine.

12. The method of claim 11, wherein the pancreatic protease inhibitor and the lipase inhibitor are administered simultaneously.

13. The method of claim 11, wherein the pancreatic protease inhibitor and the lipase inhibitor are administered sequentially.

14. A method for prevention or treatment of septic shock, the method comprising:
administering to an individual containing an intestine a therapeutic dose of a pancreatic protease inhibitor into the small intestine to inactivate a protease, thereby ameliorating shock.

15. The method of claim 14, further comprising:
administering to the individual a therapeutic dose of a lipase inhibitor into the small intestine.

16. The method of claim 15, wherein the pancreatic protease inhibitor and the lipase inhibitor are administered simultaneously.

17. The method of claim 15, wherein the pancreatic protease inhibitor and the lipase inhibitor are administered sequentially.

18. A method for reducing mortality after shock, the method comprising:
administering to an individual containing an intestine a therapeutic dose of a pancreatic protease inhibitor into the small intestine to inactivate a protease, thereby ameliorating shock.

19. The method of claim 18, further comprising:
administering to the individual a therapeutic dose of a lipase inhibitor into the small intestine.

20. The method of claim 19, wherein the pancreatic protease inhibitor and the lipase inhibitor are administered simultaneously.

21. The method of claim 19, wherein the pancreatic protease inhibitor and the lipase inhibitor are administered sequentially.
